(19) United States
(12) Patent Application Publication
(10) Pub. No.: US 2021/0000714 A1
(43) Pub. Date: Jan. 7, 2021

(54) ALCOHOL-BASED HAND CONDITIONERS FOR REPEATED USE

(71) Applicant: GOJO Industries, Inc., Akron, OH (US)

(72) Inventors: Amanda J. Copeland, Seville, OH (US); Todd J. Cartner, Mogadore, OH (US)

(21) Appl. No.: 17/027,764

(22) Filed: Sep. 22, 2020

Related U.S. Application Data
(62) Division of application No. 16/288,600, filed on Feb. 28, 2019.
(60) Provisional application No. 62/636,888, filed on Mar. 1, 2018.

(51) Int. Cl. A61K 8/34; A61Q 19/00

(52) U.S. Cl. CPC A61K 8/345; A61Q 19/007; A61Q 19/00; A61K 2800/30; A61K 2800/48; A61K 2800/5922; A61K 2800/88; A61K 8/34

(57) ABSTRACT

Alcohol-based hand conditioners are provided. The alcohol-based hand conditioners have superior aesthetics suitable for use in rapid workflow environments. They are non-greasy and low residue, and thus do not leave a sticky or tacky feeling on the hands following application.

### RELATED APPLICATIONS

This application is a divisional of U.S. Non-Provisional patent application Ser. No. 16/288,600, entitled “ALCOHOL-BASED HAND CONDITIONERS FOR REPEATED USE” and filed Feb. 28, 2019, which claims priority to and the benefit of U.S. Provisional Patent Application Ser. No. 62/636,888, entitled “ALCOHOL-BASED HAND CONDITIONERS FOR REPEATED USE” and filed Mar. 1, 2018, the entire disclosure of which is incorporated herein by reference.

### FIELD

The present disclosure relates to alcohol-based hand conditioners. More particularly, the present disclosure relates to alcohol-based hand conditioners formulated to have superior aesthetics, which makes them suitable for use in rapid workflow environments. The alcohol-based hand conditioners may be particularly useful in high-frequency hand washing environments, because they can be applied to dry skin in a conventional manner as a “leave on” product, or alternatively, they can be applied to wet skin and toweled off. The alcohol-based hand conditioners provide suitable hydration while maintaining a low percentage of solids in the composition.

### BACKGROUND

Hand hygiene is the primary measure to reduce infections. Most hospitals and health-care facilities have established hand hygiene guidelines that require frequent use of hand sanitizers and hand washes. Thus, few environments are impacted more by topical product usage than healthcare facilities.

The skin condition of healthcare workers suffers for two reasons. First, healthcare workers wash their hands many times a day, which damages the integrity of their skin. Frequent skin exposure to water and typical cleansers has profound effects on the stratum corneum structure and function. Effects include disruption of the lipid bilayer architecture to create defects or holes in the barrier. As a result, the barrier becomes more permeable, allowing irritants and microorganisms to penetrate into and through the uppermost layers of the skin. In cases of severe hand irritation, cracks or fissures (with or without bleeding) may develop indicating damage to the dermis. The skin's response to these damaging effects is immediate, but the accelerated efforts to repair the barrier and generate new stratum corneum leads to imperfect architecture, when compared to stratum corneum that is formed during the normal course of stratum corneum replacement. The rapidly-produced stratum corneum has poor water binding properties, leading to insufficient skin moisture and inadequate desquamation.

In some studies, up to 85 percent of nurses described histories of skin problems and 25 percent reported symptoms of dermatitis. Over 50 percent of inpatient nurses, and 65 percent in intensive care units had observable hand dermatitis.

The second reason that the skin of healthcare workers suffers is because hand lotions are either not available in hospitals and health-care facilities, or if they are, they are not compatible with workflow. For instance, it can be exceedingly difficult to put a medical glove on recently lotioned hands. Conventional hand lotions are frequently too greasy and/or leave behind a residue, which impedes healthcare workers' ability to perform their job functions. The incompatibility with workflow causes healthcare workers to forego using hand lotions during the course of a typical shift.

### SUMMARY

The general inventive concepts are directed to alcohol-based hand conditioners formulated to have superior aesthetics suitable for use in rapid workflow environments. The alcohol-based hand conditioners are non-greasy and low residue, and thus do not leave a sticky or tacky feeling on the hands following application. The alcohol-based hand conditioners can be applied to dry skin, or alternatively, they can be applied to wet skin and toweled off. In embodiments of the present disclosure, the alcohol-based hand conditioner composition comprises less than 25% solids.

### DETAILED DESCRIPTION

Disclosed herein are alcohol-based hand conditioners. While the present disclosure describes certain embodiments of the alcohol-based hand conditioners in detail, the present disclosure is to be considered exemplary and is not intended to be limited to the disclosed embodiments.

The terminology as set forth herein is for description of the embodiments only and should not be construed as limiting the disclosure as a whole. All references to singular characteristics or limitations of the present disclosure shall include the corresponding plural characteristic or limitation, and vice versa, unless otherwise specified or clearly implied to the contrary by the context in which the reference is made. Unless otherwise specified, “a,” “an,” “the,” and “at least one” are used interchangeably. Furthermore, as used in the description and the appended claims, the singular forms “a,” “an,” and “the” are inclusive of their plural forms, unless the context clearly indicates otherwise.

To the extent that the term “includes” or “including” is used in the description or the claims, it is intended to be inclusive in a manner similar to the term “comprising” as that term is interpreted when employed as a transitional word in a claim. Furthermore, to the extent that the term “or” is employed (e.g., A or B) it is intended to mean “A or B or both.” When the applicants intend to indicate “only A or B but not both” then the term “only A or B but not both” will be employed. Thus, use of the term “or” herein is the inclusive, and not the exclusive use.

The alcohol-based hand conditioners of the present disclosure can comprise, consist of, or consist essentially of the essential elements of the disclosure as described herein, as well as any additional or optional element described herein or which is otherwise useful in hand conditioning applications.

All percentages, parts, and ratios as used herein are by weight of the total formulation, unless otherwise specified.

All ranges and parameters, including but not limited to percentages, parts, and ratios, disclosed herein are understood to encompass any and all sub-ranges assumed and subsumed therein, and every number between the endpoints. For example, a stated range of “1 to 10” should be considered to include any and all sub-ranges beginning with a minimum value of 1 or more and ending with a maximum value of 10 or less (e.g., 1 to 6.1, or 2.3 to 9.4), and to each integer (1, 2, 3, 4, 5, 6, 7, 8, 9, and 10) contained within the range.

Any combination of method or process steps as used herein may be performed in any order, unless otherwise specified or clearly implied to the contrary by the context in which the referenced combination is made.

The general inventive concepts relate to alcohol-based hand conditioners. More particularly, the inventive concepts relate to alcohol-based hand conditioner compositions comprising an amount up to 30 wt. % of one or more C₁-C₆ alcohols, wherein the alcohol-based hand conditioner compositions comprise less than 25% solids. The alcohol-based hand conditioners can be applied to dry skin, or alternatively, they can be applied to wet skin and toweled off

In accordance with the present disclosure, the alcohol-based hand conditioner compositions comprise one or more alcohols. The term “alcohol,” as used herein, constitutes any compound containing at least one carbon bonded to at least one hydroxyl group, i.e., any molecule having the structure R₁R₂R₃C—OH, including linear or branched compounds. The identity of the “R” groups, i.e., R₁ R₂ and R₃ groups, are not particularly limited.

In accordance with the present disclosure, the alcohol-based hand conditioner compositions comprise one or more C₁-C₆ alcohols, i.e., alcohols containing 1 to 6 carbon atoms. Such alcohols may be referred to as lower alkanols. Examples of lower alkanols include, but are not limited to, methanol, ethanol, propanol, butanol, pentanol, hexanol, and isomers and mixtures thereof. In some embodiments, the one or more alcohols comprise ethanol, propanol, or butanol, or isomers or mixtures thereof In some embodiments, the one or more alcohols comprise isopropanol. In some embodiments, the one or more alcohols comprise ethanol. In some embodiments, the compositions comprise a mixture of alcohols. In some embodiments, the compositions comprise a mixture of ethanol and isopropanol. In some embodiments, the compositions comprise a mixture of isopropanol and n-propanol. In some embodiments, the one or more alcohols consist of ethanol.

In accordance with the present disclosure, the alcohol-based hand conditioner compositions comprise up to and including 30 percent by weight (wt. %) of one or more C₁-C₆ alcohols, based upon the total weight of the composition. In some embodiments, the compositions comprise less than 30 wt. % of one or more C₁-C₆ alcohols, based upon the total weight of the composition. In some embodiments, the compositions comprise up to and including 25 wt. % of one or more C₁-C₆ alcohols, based upon the total weight of the composition. In some embodiments, the compositions comprise less than 25 wt. % of one or more C₁-C₆ alcohols, based upon the total weight of the composition. In some embodiments, the compositions comprise up to and including 20 wt. % of one or more C₁-C₆ alcohols, based upon the total weight of the composition. In some embodiments, the alcohol-based hand conditioner compositions comprise from 1 to 30 wt. % of one or more C₁-C₆ alcohols, based upon the total weight of the composition. In some embodiments, the alcohol-based hand conditioner compositions comprise from 5 to 30 wt. % of one or more C₁-C₆ alcohols, based upon the total weight of the composition. In some embodiments, the alcohol-based hand conditioner compositions comprise from 5 to 25 wt. % of one or more C₁-C₆ alcohols, based upon the total weight of the composition. In some embodiments, the alcohol-based hand conditioner compositions comprise from 10 to 25 wt. % of one or more C₁-C₆ alcohols, based upon the total weight of the composition. In some embodiments, the alcohol-based hand conditioner compositions comprise from 10 to 23 wt. % of one or more C₁-C₆ alcohols, based upon the total weight of the composition. In some embodiments, the alcohol-based hand conditioner compositions comprise from 15 to 23 wt. % of one or more C₁-C₆ alcohols, based upon the total weight of the composition. In some embodiments, the alcohol-based hand conditioner compositions comprise from 18 to 22 wt. % of one or more C₁-C₆ alcohols, based upon the total weight of the composition.

In accordance with the present disclosure, the alcohol-based hand conditioner compositions may comprise one or more humectants. In some embodiments, the one or more humectants comprise propylene glycols, hexylene glycols, 1,4-dihydroxyhexane, 1,2,6-hexanetriol, sorbitol, butylene glycols, propanediols, such as methyl propane diol, dipropylene glycols, triethylene glycols, glycerin (glycerol), polyethylene glycols, ethoxydiglycols, polyethylene sorbitol, glycolic acid, glycolate salts, lactate salts, urea, hydroxyethyl urea, alpha-hydroxy acids, such as lactic acid, sodium pyrrolidone carboxylic acid, hyaluronic acid, chitin, and combinations thereof. In some embodiments, the one or more humectants comprise polyethylene glycol, glycerin, and combinations thereof. Examples of polyethylene glycol humectants include PEG-4, PEG-6, PEG-7, PEG-8, PEG-9, PEG-10, PEG-12, PEG-14, PEG-16, PEG-18, PEG-20, PEG-32, PEG-33, PEG-40, PEG-45, PEG-55, PEG-60, PEG-75, PEG-80, PEG-90, PEG-100, PEG-135, PEG-150, PEG-180, PEG-200, PEG-220, PEG-240, and PEG-800. Other exemplary humectants may include diols, C₆₋₁₀ alkane diols, and glyceryl caprylate/caprate. In some embodiments, the one or more humectants comprises one or more of glycerin, propanediol, sodium PCA, sodium lactate, betaine, hydroxyethyl urea, PEG-150, propylene glycol, butylene glycol, urea, sorbitol, hyaluronic acid, pentylene glycol, trehalose, xylitol, PCA, PEG-14, and PEG-33. In some embodiments, the one or more humectants consists of glycerin.

In accordance with the present disclosure, the alcohol-based hand conditioner compositions comprise up to and including 15 wt. % of one or more humectants, based upon the total weight of the composition. In some embodiments, the alcohol-based hand conditioner compositions comprise up to and including 12 wt. % of one or more humectants, based upon the total weight of the composition. In some embodiments, the alcohol-based hand conditioner compositions comprise up to and including 8 wt. % of one or more humectants, based upon the total weight of the composition. In some embodiments, the alcohol-based hand conditioner compositions comprise up to and including 7.5 wt. % of one or more humectants, based upon the total weight of the composition. In some embodiments, the alcohol-based hand conditioner compositions comprise greater than 2 wt. % of one or more humectants, based upon the total weight of the composition. In some embodiments, the alcohol-based hand conditioner compositions comprise greater than 3 wt. % of one or more humectants, based upon the total weight of the composition. In some embodiments, the alcohol-based hand conditioner compositions comprise greater than 5 wt. % of one or more humectants, based upon the total weight of the composition. In some embodiments, the alcohol-based hand conditioner compositions comprise from 0.01 to 15 wt. % of one or more humectants, based upon the total weight of the composition. In some embodiments, the alcohol-based hand conditioner compositions comprise from 0.1 to 10 wt. % of one or more humectants, based upon the total weight of the composition. In some embodiments, the alcohol-based hand conditioner compositions comprise from 1 to 10 wt. % of one or more humectants, based upon the total weight of the composition. In some embodiments, the alcohol-based hand conditioner compositions comprise from 2 to 10 wt. % of one or more humectants, based upon the total weight of the composition. In some embodiments, the alcohol-based hand conditioner compositions comprise from 3 to 10 wt. % of one or more humectants, based upon the total weight of the composition. In some embodiments, the alcohol-based hand conditioner compositions comprise from 5 to 10 wt. % of one or more humectants, based upon the total weight of the composition. In some embodiments, the alcohol-based hand conditioner compositions comprise from 5 to 8 wt. % of one or more humectants, based upon the total weight of the composition. In some embodiments, the alcohol-based hand conditioner compositions comprise from 5.5 to 7.5 wt. % of one or more humectants, based upon the total weight of the composition.

In accordance with the present disclosure, the alcohol-based hand conditioner compositions may comprise one or more emulsifiers. In some embodiments, the one or more emulsifiers are cationic emulsifiers. In some embodiments, the one or more emulsifiers comprise at least one wax selected from the group consisting of natural waxes and synthetic waxes, and at least one cationic polymer. In some embodiments, the one or more emulsifiers comprise one or more C12-C18 fatty acid-C2-C5 polyol esters such as glyceryl monostearate, ethylene glycol monostearate and polyethylene glycol distearate. Examples of polyethylene glycol distearates include PEG-150 distearate. Examples of cationic polymers include polyquaternium polymers, such as polyquaternium-37. The one or more emulsifiers may further comprise one or more fatty alcohols. Examples of fatty alcohols include cetearyl alcohol. In some embodiments, the one or more emulsifiers comprise a cold process formulation aid. A particular cold process formulation aid is sold under the trade name Jeesperse® ICE-T® CIS-2 by JEEN International Corporation in Fairfield, New Jersey. Jeesperse® ICE-T® CIS-2 includes PEG-150 Distearate, Cetearyl Alcohol, and Polyquaternium-37. Cold process formulation aids are further described in U.S. Patent Application Pub. No. 2016/0206543, which is incorporated by reference herein in its entirety.

In accordance with the present disclosure, the alcohol-based hand conditioner compositions may comprise up to and including 10 wt. % of one or more emulsifiers, based upon the total weight of the composition. In some embodiments, the alcohol-based hand conditioner compositions may comprise up to and including 8 wt. % of one or more emulsifiers, based upon the total weight of the composition. In some embodiments, the alcohol-based hand conditioner compositions may comprise up to and including 5 wt. % of one or more emulsifiers, based upon the total weight of the composition. In some embodiments, the alcohol-based hand conditioner compositions may comprise up to and including 3.5 wt. % of one or more emulsifiers, based upon the total weight of the composition. In some embodiments, the alcohol-based hand conditioner compositions may comprise from 0.01 to 10 wt. % of one or more emulsifiers, based upon the total weight of the composition. In some embodiments, the alcohol-based hand conditioner compositions may comprise from 0.1 to 8 wt. % of one or more emulsifiers, based upon the total weight of the composition. In some embodiments, the alcohol-based hand conditioner compositions may comprise from 0.5 to 5 wt. % of one or more emulsifiers, based upon the total weight of the composition. In some embodiments, the alcohol-based hand conditioner compositions may comprise from 1 to 4.5 wt. % of one or more emulsifiers, based upon the total weight of the composition. In some embodiments, the alcohol-based hand conditioner compositions may comprise from 2 to 4 wt. % of one or more emulsifiers, based upon the total weight of the composition.

In accordance with the present disclosure, the alcohol-based hand conditioner compositions may comprise one or more emollients. Emollients are often used to impart a smooth and soft feel to the skin without measurably affecting the skin hydration level and or the skin lipid barrier. In some embodiments, the one or more emollients comprise conditioning or moisturizing esters. In some embodiments, the one or more emollients comprise long-chain alkyl benzoates including, but not limited to, C12-15 Alkyl Benozate, C16-17 Alkyl Benzoate, Stearyl Benzoate, Isostearyl Benzoate, Ethylhexyl Benzoate and Octyldodecyl Benzoate. In some embodiments, the one or more emollients consist of C12-15 Alkyl Benozate.

In accordance with the present disclosure, the alcohol-based hand conditioner compositions may comprise up to and including 10 wt. % of one or more emollients, based upon the total weight of the composition. In some embodiments, the alcohol-based hand conditioner compositions may comprise up to and including 5 wt. % of one or more emollients, based upon the total weight of the composition. In some embodiments, the alcohol-based hand conditioner compositions may comprise up to and including 2.5 wt. % of one or more emollients, based upon the total weight of the composition. In some embodiments, the alcohol-based hand conditioner compositions may comprise from 0.01 to 10 wt. % of one or more emollients, based upon the total weight of the composition. In some embodiments, the alcohol-based hand conditioner compositions may comprise from 0.1 to 5 wt. % of one or more emollients, based upon the total weight of the composition. In some embodiments, the alcohol-based hand conditioner compositions may comprise from 0.5 to 3 wt. % of one or more emollients, based upon the total weight of the composition. In some embodiments, the alcohol-based hand conditioner compositions may comprise from 1 to 2.5 wt. % of one or more emollients, based upon the total weight of the composition.

In accordance with the present disclosure, the alcohol-based hand conditioner compositions may comprise one or more silicone glycols. Silicone glycols are generally characterized by containing one or more Si—O—Si linkages in the polymer backbone. In some embodiments, the one or more silicone glycols comprise organopolysiloxane dimethicone polyols, silicone carbinol fluids, silicone polyethers, alkylmethyl siloxanes, amodimethicones, trisiloxane ethoxylates, dimethiconols, quaternized silicone glycols, polysilicones, silicone crosspolymers, and silicone waxes. In some embodiments, the one or more silicone glycols consist of dimethicone.

In some embodiments, the one or more silicone glycols comprise dimethicone PEG-7 undecylenate, PEG-10 dimethicone, PEG-8 dimethicone, PEG-12 dimethicone, perfluorononylethyl carboxydecal PEG 10, PEG-20/PPG-23 dimethicone, PEG-11 methyl ether dimethicone, bis-PEG/PPG-20/20 dimethicone, silicone quats, PEG-9 dimethicone, PPG-12 dimethicone, fluoro PEG-8 dimethicone, PEG 23/PPG 6 dimethicone, PEG 20/PPG 23 dimethicone, PEG 17 dimethicone, PEGS/PPG3 methicone, bis PEG20 dimethicone, PEG/PPG20/15 dimethicone copolyol and sulfosuccinate blends, PEG-8 dimethicone/dimmer acid blends, PEG-8 dimethicone/fatty acid blends, PEG-8 dimethicone/cold pressed vegetable oil\polyquaternium blends, random block polymers, and mixtures thereof. In some embodiments, the one or more silicone glycols consist of PEG-12 dimethicone, PEG-10 dimethicone, PEG-8 dimethicone, and combinations thereof.

In some embodiments, the one or more silicone glycols include a compound that may be represented by the formula:

R²—Si(CH₃)₂—[O—Si(CH₃)₂]ₐ—[O—Si(CH₃)R³]_{b}—O—Si(CH₃)₂—R²

where R² and R³ independently include a methyl group or a moiety that may be represented by the formula:

—(CH₂)₃—O—(CH₂CH₂O)_{c}—[CH₂CH(CH₃)O]_{d}—(CH₂CH₂O)ₑH

with the proviso that both R² and R³ are not CH₃, where a is an integer from 3 to 21, b is an integer from 1 to 7, c is an integer from 0 to 40, d is an integer from 0 to 40, and e is an integer from 0 to 40, with the proviso that a≥3×b and that c+d+e≥5.

In accordance with the present disclosure, the alcohol-based hand conditioner compositions may comprise up to and including 10 wt. % of one or more silicone glycols, based upon the total weight of the composition. In some embodiments, the alcohol-based hand conditioner compositions may comprise up to and including 8 wt. % of one or more silicone glycols, based upon the total weight of the composition. In some embodiments, the alcohol-based hand conditioner compositions may comprise up to and including 5 wt. % of one or more silicone glycols, based upon the total weight of the composition. In some embodiments, the alcohol-based hand conditioner compositions may comprise up to and including 2 wt. % of one or more silicone glycols, based upon the total weight of the composition. In some embodiments, the alcohol-based hand conditioner compositions may comprise from 0.01 to 5 wt. % of one or more silicone glycols, based upon the total weight of the composition. In some embodiments, the alcohol-based hand conditioner compositions may comprise from 0.1 to 2 wt. % of one or more silicone glycols, based upon the total weight of the composition. In some embodiments, the alcohol-based hand conditioner compositions may comprise from 0.5 to 1.5 wt. % of one or more silicone glycols, based upon the total weight of the composition. In some embodiments, the alcohol-based hand conditioner compositions may comprise from 0.75 to 1.25 wt. % of one or more silicone glycols, based upon the total weight of the composition.

In accordance with the present disclosure, the alcohol-based hand conditioner compositions may comprise one or more deposition enhancers. Advantageously, the deposition enhancer is non-toxic, non-irritating and non-allergenic. Suitable deposition enhancers work rapidly with predictable and reproducible activity and duration. Suitably, the deposition enhancer has no significant pharmacological activity within the body, and when removed from the skin, barrier properties return rapidly and fully to normal. The deposition enhancer primarily works unidirectionally, i.e., allows therapeutic agents into the body while preventing the loss of endogenous materials from the body.

In some embodiments, the one or more deposition enhancers comprise bile salts and derivatives thereof, fatty acids and derivatives thereof, chelating agents, sulphoxides, and combinations thereof. In some embodiments, the one or more deposition enhancers comprise dimethyl sulphoxides (DMSO), DMA, DMF, 1-dodecylazacycloheptan-2-one (azone), pyrrolidones such as 2-Pyrrolidone (2P) and N-Methyl-2-Pyrrolidone (NMP), long-chain fatty acids such as oleic acid and fatty acids with a saturated alkyl chain length of about C₁₀-C₁₂, essential oils, terpenes, terpenoids, oxazolidinones such as 4-decyloxazolidin-2-one, polysorbates, sodium glycolate, sodium deoxycholate, caprylic acid, EDTA, phospholipids, pentylene glycol, ethoxydiglycol, polysorbate-polyethylenesorbitan-monolaurate, lecithin. In some embodiments, the one or more deposition enhancers comprise a hydroxy-terminated polyurethane compound chosen from polyolprepolymer-2, polyolprepolymer-14, and polyolprepolymer-15. In some embodiments, the one or more deposition enhancers consist of polyolprepolymer-2. Polyolprepolymer-2 is sometimes referred to as PPG-12/SMDI copolymer.

In accordance with the present disclosure, the alcohol-based hand conditioner compositions may comprise up to and including 10 wt. % of one or more deposition enhancers, based upon the total weight of the composition. In some embodiments, the alcohol-based hand conditioner compositions may comprise up to and including 8 wt. % of one or more deposition enhancers, based upon the total weight of the composition. In some embodiments, the alcohol-based hand conditioner compositions may comprise up to and including 5 wt. % of one or more deposition enhancers, based upon the total weight of the composition. In some embodiments, the alcohol-based hand conditioner compositions may comprise up to and including 1 wt. % of one or more deposition enhancers, based upon the total weight of the composition. In some embodiments, the alcohol-based hand conditioner compositions may comprise up to and including 0.75 wt. % of one or more deposition enhancers, based upon the total weight of the composition. In some embodiments, the alcohol-based hand conditioner compositions may comprise up to and including 0.5 wt. % of one or more deposition enhancers, based upon the total weight of the composition. In some embodiments, the alcohol-based hand conditioner compositions may comprise from 0.01 to 5 wt. % of one or more deposition enhancers, based upon the total weight of the composition. In some embodiments, the alcohol-based hand conditioner compositions may comprise from 0.1 to 2 wt. % of one or more deposition enhancers, based upon the total weight of the composition. In some embodiments, the alcohol-based hand conditioner compositions may comprise from 0.3 to 0.75 wt. % of one or more deposition enhancers, based upon the total weight of the composition.

In accordance with the present disclosure, the alcohol-based hand conditioner compositions may comprise one or more skin conditioners, thickeners, or both. The terms “skin conditioners” and “thickeners” are not intended to be limiting, as various ingredients may perform both a skin conditioning and a thickening function.

Non-limiting examples of suitable skin conditioners include aloe, vitamin E, vitamin E acetate (tocopheryl acetate), Vitamin B3 (niacinamide), C₆₋₁₀ alkane diols, glyceryl caprylate/caprate, sodium salt of pyroglutamic acid (sodium PCA), PEG-7 glyceryl cocoate, coco-glucoside and/or glyceryl oleate (Lamisoft® PO), and polyquaterniums, such as polyquaternium-10 and polyquaternium-39. Any of these skin conditioners may or may not likewise act as a thickener in the alcohol-based hand conditioner compositions. In some embodiments, the one or more skin conditioners and thickeners comprise polyquaternium-39. In some embodiments, the one or more skin conditioners and thickeners comprise one or more diols. In some embodiments, the one or more skin conditioners and thickeners comprise one or more C₆₋₁₀ alkane diols, i.e., diols having a carbon chain length of 6 to 10. In some embodiments, the diol comprises a straight chain diol. In some embodiments, the diol may be 1,2-hexanediol, 1,2-octanediol, 1,9-nonanediol, 1,2-decanediol, 1,10-decanediol, or a mixture thereof 1,2-octanediol is sometimes referred to as caprylyl glycol.

Non-limiting examples of suitable thickeners include polysaccharaide thickeners, such as starch, vegetable gums, pectin, and guar. Other non-limiting examples of suitable thickeners include polyacrylate thickeners such as those conventionally available and/or known in the art. Examples of polyacrylate thickeners include carbomers, acrylates/C10-30 alkyl acrylate crosspolymers, copolymers of acrylic acid and alkyl (C5-C10) acrylate, copolymers of acrylic acid and maleic anhydride, and mixtures thereof. Polyacrylate thickeners are further described in U.S. Patent Application Publication No. 2010/0317743 A1, which is hereby incorporated by reference. In some embodiments, the one or more skin conditioners and thickeners comprise starch.

Other non-limiting examples of suitable thickeners that may or may not likewise act as skin conditioners include stearyl alcohol, cationic hydroxy ethyl cellulose, hydroxy propyl methyl cellulose, hydroxy propyl cellulose, chitosan pyrrolidone carboxylate, behenyl alcohol, zinc stearate, and emulsifying waxes, including but not limited to Incroquat and Polawax, polymers of acrylic acid, a resin such as Carbopol® ETD 2020, guar gum, acacia, acrylates/steareth-20 methacrylate copolymer, agar, algin, alginic acid, ammonium acrylate copolymers, ammonium alginate, ammonium chloride, ammonium sulfate, amylopectin, attapulgite, bentonite, C9-15 alcohols, calcium acetate, calcium alginate, calcium carrageenan, calcium chloride, caprylic alcohol, carbomer 910, carbomer 934, carbomer 934P, carbomer 940, carbomer 941, carboxymethyl hydroxyethyl cellulose, carboxymethyl hydroxypropyl guar, carrageenan, cellulose, cellulose gum, cetearyl alcohol, cetyl alcohol, corn starch, damar, dextrin, dibenzlidine sorbitol, ethylene dihydrogenated tallowamide, ethylene diolamide, ethylene distearamide, gelatin, guar gum, guar hydroxypropyltrimonium chloride, hectorite, hyaluronic acid, hydrated silica, hydroxybutyl methylcellulose, hydroxy ethylcellulose, hydroxyethyl ethylcellulose, hydroxyethyl stearamide-MIPA, isocetyl alcohol, isostearyl alcohol, karaya gum, kelp, lauryl alcohol, locust bean gum, magnesium aluminium silicate, magnesium silicate, magnesium trisilicate, methoxy PEG-22/dodecyl glycol copolymer, methylcellulose, microcrystalline cellulose, montmorillonite, myristyl alcohol, oat flour, oleyl alcohol, palm kernel alcohol, pectin, PEG-2M, PEG-5M, polyacrylic acid, polyvinyl alcohol, potassium alginate, potassium aluminium polyacrylate, potassium carrageenan, potassium chloride, potassium sulfate, potato starch, propylene glycol alginate, sodium acrylate/vinyl alcohol copolymer, sodium carboxymethyl dextran, sodium carrageenan, sodium cellulose sulfate, sodium chloride, sodium polymethacylate, sodium silicoaluminate, sodium sulfate, stearalkonium bentonite, stearalkonium hectorite, stearyl alcohol, tallow alcohol, TEA-hydrochloride, tragacanth gum, tridecyl alcohol, tromethamine magnesium aluminium silicate, wheat flour, wheat starch, xanthan gum, abietyl alcohol, acrylinoleic acid, aluminum behenate, aluminum caprylate, aluminum dilinoleate, aluminum salts, such as distearate, and aluminum isostearates, beeswax, behenamide, butadiene/acrylonitrile copolymer, C29-70 acid, calcium behenate, calcium stearate, candelilla wax, carnauba, ceresin, cholesterol, cholesterol hydroxystearate, coconut alcohol, copal, diglyceryl stearate malate, dihydroabietyl alcohol, dimethyl lauramine oleate, dodecanoic acid/cetearyl alcohol/glycol copolymer, erucamide, ethylcellulose, glyceryl triacetyl hydroxystearate, glyceryl tri-acetyl ricinolate, glycol dibehenate, glycol di-octanoate, glycol distearate, hexanediol distearate, hydrogenated C6-14 olefin polymers, hydrogenated castor oil, hydrogenated cottonseed oil, hydrogenated lard, hydrogenated menhaden oil, hydrogenated palm kernel glycerides, hydrogenated palm kernel oil, hydrogenated palm oil, hydrogenated polyisobutene, hydrogenated soybean oil, hydrogenated tallow amide, hydrogenated tallow glyceride, hydrogenated vegetable glyceride, hydrogenated vegetable oil, Japan wax, jojoba wax, lanolin alcohol, shea butter, lauramide, methyl dehydroabietate, methyl hydrogenated rosinate, methyl rosinate, methylstyrene/vinyltoluene copolymer, microcrystalline wax, montan acid wax, montan wax, myristyleicosanol, myristyloctadecanol, octadecene/maleic anhyrdine copolymer, octyldodecyl stearoyl stearate, oleamide, oleostearine, ouricury wax, oxidized polyethylene, ozokerite, paraffin, pentaerythrityl hydrogenated rosinate, pentaerythrityl tetraoctanoate, pentaerythrityl rosinate, pentaerythrityl tetraabietate, pentaerythrityl tetrabehenate, pentaerythrityl tetraoleate, pentaerythrityl tetrastearate, ophthalmic anhydride/glycerin/glycidyl decanoate copolymer, ophthalmic/trimellitic/glycols copolymer, polybutene, polybutylene terephthalate, polydipentene, polyethylene, polyisobutene, polyisoprene, polyvinyl butyral, polyvinyl laurate, propylene glycol dicaprylate, propylene glycol dicocoate, propylene glycol diisononanoate, propylene glycol dilaurate, propylene glycol dipelargonate, propylene glycol distearate, propylene glycol diundecanoate, PVP/eiconsene copolymer, PVP/hexadecene copolymer, rice bran wax, stearlkonium bentonite, stearalkonium hectorite, stearamide, stearamide DEA-distearate, stearamide DIBA-stearate, stearamide MEA-stearate, stearone, stearyl erucamide, stearyl stearate, stearyl stearoyl stearate, synthetic beeswax, synthetic wax, trihydroxystearin, triisononanoin, triisostearin, tri-isostearyl trilinoleate, trilaurin, trilinoleic acid, trilinolein, trimyristin, triolein, tripalmitin, tristearin, zinc laurate, zinc myristate, zinc neodecanoate, zinc rosinate, and mixtures thereof. In some embodiments, the one or more skin conditioners and thickeners comprise shea butter.

In accordance with the present disclosure, the alcohol-based hand conditioner compositions may comprise up to and including 15 wt. % of one or more skin conditioners and thickeners, based upon the total weight of the composition. In some embodiments, the alcohol-based hand conditioner compositions may comprise up to and including 8 wt. % of one or more skin conditioners and thickeners, based upon the total weight of the composition. In some embodiments, the alcohol-based hand conditioner compositions may comprise up to and including 5 wt. % of one or more skin conditioners and thickeners, based upon the total weight of the composition. In some embodiments, the alcohol-based hand conditioner compositions may comprise from 0.01 to 7 wt. % of one or more skin conditioners and thickeners, based upon the total weight of the composition. In some embodiments, the alcohol-based hand conditioner compositions may comprise from 0.1 to 5 wt. % of one or more skin conditioners and thickeners, based upon the total weight of the composition. In some embodiments, the alcohol-based hand conditioner compositions may comprise from 0.5 to 4 wt. % of one or more skin conditioners and thickeners, based upon the total weight of the composition.

In accordance with the present disclosure, the alcohol-based hand conditioner compositions comprise water quantum sufficit (q.s.). In some embodiments, the alcohol-based hand conditioner compositions comprise at least 30 weight percent (wt. %) water. In some embodiments, the alcohol-based hand conditioner compositions comprise at least 40 wt. % water. In some embodiments, the alcohol-based hand conditioner compositions comprise at least 50 wt. % water. In some embodiments, the alcohol-based hand conditioner compositions comprise at least 60 wt. % water. In some embodiments, the alcohol-based hand conditioner compositions comprise from 30 to 80 wt. % water. In some embodiments, the alcohol-based hand conditioner compositions comprise from 40 to 75 wt. % water. In some embodiments, the alcohol-based hand conditioner compositions comprise from 50 to 75 wt. % water. In some embodiments, the alcohol-based hand conditioner compositions comprise from 55 to 65 wt. % water. More or less water may be required in certain instances, depending particularly on other ingredients and/or the amounts thereof employed.

The alcohol-based hand conditioner compositions of the present invention can further comprise a wide range of optional additives, with the proviso that they do not deleteriously affect the aesthetics of the compositions. The CTFA International Cosmetic Ingredient Dictionary and Handbook, Eleventh Edition 2005, and the 2004 CTFA International Buyer's Guide, both of which are incorporated by reference herein in their entirety, describe a wide variety of non-limiting cosmetic and pharmaceutical ingredients commonly used in the skin care industry, that are suitable for use in the compositions of the present invention. Nonlimiting examples of functional classes of ingredients are described at page 537 of this reference. Examples of these functional classes include: abrasives, anti-acne agents, anticaking agents, antioxidants, antipruritics, binders, biological additives, bulking agents, chelating agents, chemical additives, colorants, cosmetic astringents, cosmetic biocides, denaturants, drug astringents, emulsifiers, external analgesics, film formers, fragrance components, humectants, opacifying agents, plasticizers, preservatives (sometimes referred to as antimicrobials), propellants, reducing agents, skin bleaching agents, skin-conditioning agents (emollient, miscellaneous, and occlusive), skin protectants, solvents, foam boosters, hydrotropes, solubilizing agents, suspending agents (nonsurfactant), sunscreen agents, ultraviolet light absorbers, rheology modifying agents, including salts and polymers, detackifiers, and viscosity increasing agents (aqueous and nonaqueous). Examples of other functional classes of materials that may be useful herein include solubilizing agents, sequestrants, keratolytics, topical active ingredients, and the like. In some embodiments, the alcohol-based hand conditioner compositions comprise antioxidants, including, but not limited to, avenanthramides. Avenanthramides are described in detail in the reference U.S. Pat. Appln. Pub. No. 2015/0258003, which is incorporated herein by reference in its entirety.

In accordance with the present disclosure, the alcohol-based hand conditioner compositions may comprise up to and including 5 wt. % of one or more additives, based upon the total weight of the composition. In some embodiments, the alcohol-based hand conditioner compositions comprise up to and including 2 wt. % of one or more additives, based upon the total weight of the composition. In some embodiments, the alcohol-based hand conditioner compositions may comprise up to and including 1 wt. % of one or more additives, based upon the total weight of the composition. In some embodiments, the alcohol-based hand conditioner compositions may comprise from 0.001 to 2 wt. % of one or more additives, based upon the total weight of the composition. In some embodiments, the alcohol-based hand conditioner compositions may comprise from 0.01 to 1 wt. % of one or more additives, based upon the total weight of the composition. In some embodiments, the alcohol-based hand conditioner compositions may comprise from 0.05 to 0.5 wt. % of one or more additives, based upon the total weight of the composition.

In accordance with the present disclosure, the alcohol-based hand conditioners of the present invention may exclude surfactants. In some embodiments, the alcohol-based hand conditioners contain less than 5 wt. %, or less than 3 wt. %, or less than 0.5 wt. %, or less than 0.1 wt. % surfactants. In some embodiments, the alcohol-based hand conditioners are devoid of surfactants. Specifically, the alcohol-based hand conditioners may be devoid of anionic, nonionic, cationic, zwitterionic, fatty acid surfactants, or other commonly understood surfactants and surfactant systems used in cleansing compositions.

In accordance with the present disclosure, the alcohol-based hand conditioners of the present invention may be formulated as lotions. As is known in the art, lotions include oil-in-water emulsions as well as water-in-oil emulsions, oil-water-oil, and water-oil-water.

The alcohol-based hand conditioners may have a viscosity of from 5,000 to 50,000 cps at 20° C. In some embodiments, the alcohol-based hand conditioners may have a viscosity of from 10,000 to 40,000 cps at 20° C. In some embodiments, the alcohol-based hand conditioners may have a viscosity of from 15,000 to 30,000 cps at 20° C. The viscosity of samples in accordance with the present disclosure were measured using very low shear viscometer such as Brookfield LVDV-I+ viscometer and T spindles with a heliopath adapter. Unless stated otherwise, the measured viscosity is that of the final composition.

The disclosed alcohol-based hand conditioners provide suitable hydration while maintaining a low percentage of solids in the composition. In accordance with the present disclosure, the percentage of solids in the composition is less than 25%. In some embodiments, the percentage of solids in the composition is less than 20%. In some embodiments, the percentage of solids in the composition is less than 18%. In some embodiments, the percentage of solids in the composition is less than 15%. In some embodiments, the percentage of solids in the composition is less than 10%. In some embodiments, the percentage of solids in the composition is from 1 to 25%. In some embodiments, the percentage of solids in the composition is from 1 to 20%. In some embodiments, the percentage of solids in the composition is from 1 to 18%. In some embodiments, the percentage of solids in the composition is from 1 to 15%. In some embodiments, the percentage of solids in the composition is from 1 to 10%. In some embodiments, the percentage of solids in the composition is from 5 to 25%. In some embodiments, the percentage of solids in the composition is from 5 to 20%. In some embodiments, the percentage of solids in the composition is from 5 to 18%. In some embodiments, the percentage of solids in the composition is from 5 to 15%. In some embodiments, the percentage of solids in the composition is from 5 to 10%.

The ability to achieve superior aesthetics through the inclusion of alcohol in the inventive hand conditioners was particularly surprising. It is not intuitive to include alcohol in a lotion format, as one would expect a composition including alcohol to lose the appeal and aesthetics that users expect from a lotion. Moreover, it is often thought that alcohol is drying when applied to the skin, especially in leave-on formats. However, the disclosed alcohol-based hand conditioners achieve significant conditioning benefits with the inclusion of up to 30 wt. % alcohol, in addition to achieving improved aesthetics, including spreadability and rapid drying time.

A further advantage of the disclosed alcohol-based hand conditioners is the use of alcohol in place of traditional preservatives. In some embodiments, the disclosed alcohol-based hand conditioners are devoid of parabens. Traditional preservatives, including parabens, are often irritating to the skin. This irritation is especially significant in a leave-on formulation, because the traditional preservatives that may cause skin irritation are not rinsed off. Moreover, in facilities or professions where the use of gloves is prevalent, the irritation potential of traditional preservatives such as parabens left on the hands is exacerbated under the occlusion of gloves. In contrast, the alcohol component of the disclosed alcohol-based hand conditioners evaporates quickly, which both improves the aesthetics and minimizes irritation potential.

Another surprising feature of the disclosed alcohol-based hand conditioners is the ability to provide suitable hydration while maintaining a low percentage of solids in the composition. The low percentage of solids contributes to the superior aesthetics of the inventive hand conditioners, and makes them suitable for use in rapid workflow environments. The alcohol-based hand conditioners are non-greasy and low residue, and thus do not leave a sticky or tacky feeling on the hands following application. As shown in Table 1, other commercially available lotions require a high percentage of solids to provide suitable hydration:

| | Medline | Curel Hydra | |
|---|---|---|---|
| | Remedy | Therapy | Jergens |
| | Skin Repair | Wet Skin | Wet Skin |
| | Cream** | Moisturizer** | Moisturizer** |
| Percent Solids* | 16.55 | 35.82 | 38.22 |

*The percent solids of each lotion was measured using the Ohaus ® Moisture Balance, which weighs and heats the formula to determine the percentage of solids, or nonvolatile components, within the product.
**The referenced products are commercially available as the following: Medline Remedy ® With Olivamine ® Skin Repair Cream; Curél ® Hydra Therapy Wet Skin Moisturizer; and Jergens ® Wet Skin Moisturizer.

Additionally, the disclosed alcohol-based hand conditioners are particularly useful in high-frequency hand washing environments because they can be applied to dry skin in a conventional manner as a “leave on” product, or alternatively, they can be applied to wet skin and toweled off. For example, a two-step skin care regimen may include a first step of washing, wiping, or sanitizing the hands, and a second step of applying the disclosed alcohol-based hand conditioners to the dry or wet hands immediately thereafter. One particular application in high-frequency hand washing environments may be to wash, wipe, or sanitize the hands, then to apply the disclosed alcohol-based hand conditioners to the hands while still wet, and thereafter to pat the hands dry. It is envisioned that cleansers or sanitizers may be packaged or otherwise distributed in a kit together with the disclosed alcohol-based hand conditioners, to facilitate a two-step skin care regimen.

Another advantage of the disclosed alcohol-based hand conditioners is the ability to provide suitable hydration in the absence of the conventional moisturizing ingredient petrolatum. The disclosed alcohol-based hand conditioner compositions may be devoid of hydrocarbon emollients, including hydrocarbon oils and petrolatum. Compositions excluding hydrocarbon emollients do not have a base odor like petrolatum-based products, and thus the compositions may be fragrance-free, or may more easily be formulated with a pleasing fragrance. Further, petrolatum has a distinct greasy after-feel. Thus, the disclosed alcohol-based hand conditioner compositions are non-greasy, enabling ease of gloving, interaction with equipment, and increased product usage throughout a healthcare worker's shift.

Disclosed herein is a hand conditioner composition comprising: an amount up to 30 wt. % of one or more C₁-C₆ alcohols, based upon the total weight of the hand conditioner composition; greater than 2 wt. % of one or more humectants; and water, wherein the hand conditioner composition comprises less than 25% solids. In some embodiments, the hand conditioner composition comprises from 10 to 25 wt. % of one or more C₁-C₆ alcohols, based upon the total weight of the hand conditioner composition. In some embodiments, the one or more C₁-C₆ alcohols are selected from the group consisting of methanol, ethanol, propanol, butanol, pentanol, hexanol, and isomers of any thereof, or may consist of ethanol. In some embodiments, the one or more humectants are selected from the group consisting of propylene glycols, hexylene glycols, 1,4-dihydroxyhexane, 1,2,6-hexanetriol, sorbitol, butylene glycols, propanediol s, dipropylene glycols, triethylene glycols, glycerin, polyethylene glycols, ethoxydiglycols, polyethylene sorbitol. In some embodiments, the humectant comprises glycerin in an amount from 3 to 10 wt. %, based upon the total weight of the hand conditioner composition. In some embodiments, the hand conditioner composition further comprises one or more emollients, emulsifiers, deposition enhancers, silicone glycols, thickeners, skin conditioners, and combinations thereof In some embodiments, the hand conditioner composition is devoid of petrolatum. In some embodiments, the hand conditioner composition comprises from 1 to 15% solids, or less than 10% solids.

Disclosed herein is a kit comprising a cleanser and a hand conditioner composition, the hand conditioner composition comprising: an amount up to 30 wt. % of one or more C₁-C₆ alcohols, based upon the total weight of the hand conditioner composition; greater than 2 wt. % of one or more humectants; and water, wherein the hand conditioner composition comprises less than 25% solids.

Disclosed herein is a method of moisturizing skin, the method comprising: cleansing the skin with water; and applying a hand conditioner to the skin cleansed with water, the hand conditioner comprising: an amount up to 30 wt. % of one or more C₁-C₆ alcohols, based upon the total weight of the hand conditioner composition; greater than 2 wt. % of one or more humectants; and water, wherein the hand conditioner composition comprises less than 25% solids. In some embodiments, the hand conditioner composition comprises from 10 to 25 wt. % of one or more C₁-C₆ alcohols, based upon the total weight of the hand conditioner composition. In some embodiments, the hand conditioner composition comprises from 1 to 15% solids, or less than 10% solids.

Disclosed herein is a lotion composition comprising: from 10 to 25 wt. % ethanol, based upon the total weight of the lotion composition; from 2 to 10 wt. % glycerin, based upon the total weight of the lotion composition; one or more emollients; one or more deposition enhancers; one or more emulsifiers; and water, wherein the lotion composition comprises from 1 to 15% solids. In some embodiments, the one or more emulsifiers comprise a cold process formulation aid. In some embodiments, the lotion composition has a viscosity of from 5,000 to 50,000 cps at 20° C. In some embodiments, the lotion composition comprises less than 10% solids.

Disclosed herein is a kit comprising a cleanser and a lotion composition, the lotion composition comprising: from 10 to 25 wt. % ethanol, based upon the total weight of the lotion composition; from 2 to 10 wt. % glycerin, based upon the total weight of the lotion composition; one or more emollients; one or more deposition enhancers; one or more emulsifiers; and water, wherein the lotion composition comprises from 1 to 15% solids.

The general inventive concepts have been described above both generally and with regard to various specific exemplary embodiments. Although the general inventive concepts have been set forth in what are believed to be exemplary illustrative embodiments, a wide variety of alternatives will be apparent to those of skill in the art from reading this disclosure. The general inventive concepts are not otherwise limited, except for those instances when presented in specific claims.

What is claimed is:

1. A method of moisturizing skin, the method comprising:
cleansing the skin with water; and
applying a hand conditioner composition to the skin cleansed with water, the hand conditioner composition comprising:
an amount up to 30 wt. % of one or more C₁-C₆ alcohols, based upon the total weight of the hand conditioner composition;
greater than 2 wt. % of one or more humectants; and
water,
wherein the hand conditioner composition comprises less than 25% solids, wherein the hand conditioner composition is devoid of anionic and zwitterionic surfactants, and wherein the hand conditioner composition is devoid of hydrocarbon oils.

2. The method of claim 1, wherein the hand conditioner composition comprises from 15 to 25 wt. % of one or more C₁-C₆ alcohols, based upon the total weight of the hand conditioner composition.

3. The method of claim 1, wherein the one or more C₁-C₆ alcohols comprises ethanol.

4. The method of claim 1, wherein the hand conditioner composition comprises from 1 to 15% solids.

5. The method of claim 1, wherein the hand conditioner composition comprises less than 10% solids.

6. The method of claim 1, wherein the humectant comprises glycerin in an amount from 3 to 10 wt. %, based upon the total weight of the hand conditioner composition.

7. The method of claim 1, wherein the hand conditioner composition is devoid of petrolatum.

8. The method of claim 1, further including the step of drying the skin after applying the hand conditioner to the skin.

9. A method of moisturizing skin, the method comprising:
cleansing the skin with water; and
applying a hand conditioner composition to the skin cleansed with water, the hand conditioner composition comprising:
an amount up to 30 wt. % of one or more C₁-C₆ alcohols, based upon the total weight of the hand conditioner composition;
greater than 2 wt. % of one or more humectants; and
water,
wherein the hand conditioner composition comprises less than 25% solids.

10. The method of claim 9, wherein the hand conditioner composition comprises from 15 to 25 wt. % of one or more C₁-C₆ alcohols, based upon the total weight of the hand conditioner composition.

11. The method of claim 9, wherein the one or more C₁-C₆ alcohols are selected from the group consisting of methanol, ethanol, propanol, butanol, pentanol, hexanol, and isomers of any thereof.

12. The method of claim 11, wherein the one or more C₁-C₆ alcohols consists of ethanol.

13. The method of claim 9, wherein the hand conditioner composition comprises from 1 to 15% solids.

14. The method of claim 9, wherein the hand conditioner composition comprises less than 10% solids.

15. The method of claim 9, wherein the humectant comprises glycerin in an amount from 3 to 10 wt. %, based upon the total weight of the hand conditioner composition.

16. The method of claim 9, wherein the hand conditioner composition is devoid of petrolatum.

17. The method of claim 9, wherein the hand conditioner composition is devoid of anionic and zwitterionic surfactants.

18. The method of claim 9, wherein the hand conditioner composition is devoid of hydrocarbon oils.

19. The method of claim 9, further including the step of drying the skin after applying the hand conditioner to the skin.

20. A method of moisturizing hands, the method comprising:
cleansing the hands with water; and
applying a hand conditioner composition to the hands cleansed with water, the hand conditioner composition comprising:
from 15 to 23 wt. % of one or more C₁-C₆ alcohols, based upon the total weight of the hand conditioner composition;
greater than 2 wt. % of one or more humectants; and
water,
wherein the hand conditioner composition comprises less than 25% solids, and
wherein the hand conditioner composition is devoid of anionic and zwitterionic surfactants.
